(19) United States
(12) Patent Application Publication
(10) Pub. No.: US 2018/0266991 A1
(43) Pub. Date: Sep. 20, 2018

(54) MAGNETO-IMPEDANCE (MI) SENSORS EMPLOYING CURRENT CONFINEMENT AND EXCHANGE BIAS LAYER(S) FOR INCREASED SENSITIVITY

(71) Applicant: QUALCOMM Incorporated, San Diego, CA (US)

(72) Inventors: Jimmy Jianan Kan, San Diego, CA (US); Peiyuan Wang, San Diego, CA (US); Chando Park, Irvine, CA (US); Seung Hyuk Kang, San Diego, CA (US)

(21) Appl. No.: 15/459,556

(22) Filed: Mar. 15, 2017

(51) Int. Cl. G01N 27/74; G01R 33/12; G01R 33/09; G01N 33/543; G01N 27/12

(52) U.S. Cl. CPC G01N 27/745; G01R 33/1269; G01N 27/122; G01N 33/5438; G01R 33/09

(57) ABSTRACT

Magneto-impedance (MI) sensors employing current confinement and exchange bias layer(s) for increased MI sensitivity are disclosed. MI sensors may be used as biosensors to detect biological materials. The sensing by the MI devices is based on a giant magneto-impedance (GMI) effect, which is very sensitive to a magnetic field. The GMI effect is a change in impedance of a magnetic material resulting from a change in skin depth of the magnetic material as a function of an external direct current (DC) magnetic field applied to the magnetic material and an alternating current (AC) current flowing through the magnetic material (or adjacent conductive materials). Thus, this change in impedance resulting from a magnetic stray field generated by magnetic nanoparticles can be detected in lower concentrations and measured to determine the amount of magnetic nanoparticles present, and thus the target analyte of interest.

### BACKGROUND

### I. Field of the Disclosure

The technology of the disclosure relates generally to magneto-impedance (MI) devices, and more particularly to use of MI devices as MI sensors, such as biosensors, for detecting the presence of magnetic nanoparticles.

### II. Background

It may be desired in health care and other related fields to be able to detect the presence of a target analyte in a biological sample for diagnosing, monitoring, and/or maintaining health and wellness. Detecting target analytes may also be desired for performing certain health care related applications, such as human genotyping, bacteriological screening, and biological and pharmacological research. In this regard, biosensing systems can be employed to detect the presence of a target analyte in a biological sample for such applications. Biosensors are employed in biosensing systems to detect the presence of target analytes. A biosensor consists of two (2) components: a bioreceptor and a transducer. A bioreceptor is a biomolecule that recognizes the target analyte. The transducer converts the recognition event of the target analyte into a measurable signal based on a change that occurs from the bioreceptor in reaction in the presence of the target analyte. For example, a biosensor could be provided that measures glucose concentration in a blood sample by simply dipping the biosensor in the sample. This is in contrast to a conventional assay in which many steps are used and wherein each step may require a reagent to treat the sample. The simplicity and the speed of measurement is a main advantage of a biosensor. Biosensors can be provided in many different forms including non-invasive, in vitro, transcutaneous, ingested (e.g., a pill), and as a wearable or surgically implanted device.

FIG. 1 illustrates an exemplary biosensing system 100 that employs a biosensor for detecting a presence and/or properties of a biological sample. A biological sample 102 to be tested is obtained or prepared. The biological sample 102 is a sensitive biological element (e.g., tissue, microorganisms, organelles, cell receptors, enzymes, antibodies, nucleic acids, etc.) and is a biologically derived material or biomimetic component that interacts (binds or recognizes) with the target analyte under study. Examples of biological samples include cell cultures, human samples, food samples, and environmental samples. The biological sample 102 is then processed to separate a target analyte 104 of interest (e.g., a certain molecule, nucleotide, protection, and metal ion). The target analyte 104 is then introduced to target bioreceptors 106 that are designed to interact with the specific target analyte 104 of interest to produce an effect measurable by a transducer. Unbound analytes are washed away.

Magnetic labeling can be used to detect a target analyte of interest in biodetection. Magnetic labeling is effective for biodetection, because of the potential for larger signal-to-noise ratios (SNRs) in detection. For example, body liquids and tissues are not strongly magnetic by nature, which helps to improve the detection limit of magnetic biosensors and eliminate interference effects. Magnetic labeling for biodetection can also be applied to detect many different types of biomolecules beyond the conventional chemical/optical/fluorescence techniques with a large, linear dynamic range.

In this regard, one type of biosensor that has been developed to detect a target analyte of interest is a magneto-resistive (MR) biosensor. MR biosensors include a transducer that is configured to recognize a magnetic field change as a function of a sensed resistance. In this regard, as shown in FIG. 1, superparamagnetic nanoparticles 108 (hereinafter “magnetic nanoparticles 108”) can be introduced and captured by the target bioreceptors 106 that are bound to the target analyte 104. The magnetic nanoparticles 108 can then be introduced to an MR sensor 110 to detect the presence of the magnetic nanoparticles 108. The MR sensor 110 measures the magnetic field change as a result of introduction of the magnetic nanoparticles 108 as function of a change in resistance. The MR sensor 110 generates a signal 112 representing this change in resistance that can be analyzed by a sensing circuit 114 to determine the presence of the target analyte 104 in the target bioreceptor 106.

One type of MR sensing technology that can be employed in biosensing applications is a giant magneto-resistive (GMR) biosensor, such as a GMR sensor 200 shown in FIG. 2. The GMR sensor 200 can be fabricated using standard complementary metal-oxide semiconductor (CMOS) fabrication technology. The GMR effect of the GMR sensor 200 originates from its spin-dependent scattering, which depends on a relative spin of a carrier and scattering site. In this regard, the GMR sensor 200 includes a GMR device 202 that includes a pinned layer 204, a non-magnetic metal spacer 206, and a free layer 208 that has a variable magnetization. The pinned layer 204 is formed on a substrate 210 and is comprised of a ferromagnetic material (e.g., a Cobalt (Co) material) that has a fixed horizontal magnetization in the X direction, which is in-plane to the GMR device 202. The metal spacer 206, such as a Copper (Cu) spacer, is disposed above the pinned layer 204. The free layer 208 is disposed above the metal spacer 206. The free layer 208 has a magnetization that can rotate freely based on the change in a magnetic stray field 212 applied to the free layer 208. The magnetic stray field 212 is provided by the magnetization of magnetic nanoparticles 214 passing in a channel 216 (e.g., a microfluidic channel) in the GMR sensor 200, thereby forming a biological active area that is captured by bioreceptors bound to a target analyte to be detected. The channel 216 may be formed in a passivation layer 218 of a biochip 220 above a metal cap layer 222 such that the channel 216 is externally accessible from the internal components of the biochip 220 that forms a microfluidic device. For example, the magnetic nanoparticles 214 may be in a fluid form that is disposed in the channel 216. An external magnetic field 224, such as from an external coil, is applied longitudinal or perpendicular to the channel 216 to align and saturate the magnetic moments of the magnetic nanoparticles 214. Thus, when the magnetic nanoparticles 214 pass in the channel 216 above the free layer 208 of a first polarity, the magnetic stray field 212 of the magnetic nanoparticles 214 induces a change in the magnetic moment in the free layer 208. For example, the magnetic stray field 212 may only disturb the magnetic moment of the free layer 208 such that the magnetic moment rotates as little as one (1) degree. This change in the magnetic moment of the free layer 208 causes a change in resistance of the GMR device 202. This change in resistance resulting from disturbing the magnetic moment of the free layer 208 can be determined based on sensing a voltage change in the GMR device 202. For example, a sense current I_{S}_{₁} can be directed to flow through the metal spacer 206 and the free layer 208, and between metal lines 226(1), 226(2) to measure the voltage across the metal lines 226(1), 226(2) based on the resistance of the GMR device 202 according to Ohm's law.

Detection of magnetic labels using conventional magnetic sensors such as the GMR sensor 200 in FIG. 2 may not be possible at the lowest serum concentrations. For example, GMR-based biosensors may only be capable of detecting a magnetic stray field from a magnetic nanoparticle between 0.01 to 20 Oersted (Oe). As another example, a tunnel magneto-resistive (TMR)-based biosensor may be employed that can tunnel current between two (2) ferromagnetic layers and whose resistance changes as a function of the angle of magnetization between the two (2) ferromagnetic layers. However, a TMR-based biosensor may only be capable of detecting a magnetic stray field from a magnetic nanoparticle between 0.005 to 100 Oe. However, a magnetic nanoparticle that captures the target analyte of interest may only induce a magnetic stray field lower than 0.005 Oe in field strength.

### SUMMARY OF THE DISCLOSURE

Aspects disclosed herein include magneto-impedance (MI) sensors employing current confinement and exchange bias layer(s) for increased MI sensitivity. For example, these MI sensors may be used as biosensors to detect the presence of biological materials. The MI sensing by the MI devices is based on a giant magneto-impedance (GMI) effect. The GMI effect is much more sensitive to a magnetic field than, for example, a giant magneto-resistive (GMR) effect. As an example, a GMI device may be capable of detecting a magnetic stray field down to 10⁻⁸ Oerstead (Oe), to a sensitivity of 100%/Oe. The GMI effect is a change in impedance of a magnetic material resulting from a change in skin depth of the magnetic material as a function of an external direct current (DC) magnetic field applied to the magnetic material and an alternating current (AC) current flowing through the magnetic material. Skin depth is the distance between the surface of a conductor and the point within the conductor where the amplitude of an AC current reduces to a defined percentage (e.g., 37%) of its original value at the surface of the conductor. Skin depth of a conductor is an inverse function of the permeability of the conductor and the frequency of the AC current flowing through the conductor. The permeability of a ferromagnetic material conductor depends on the direction and magnitude of the external magnetic field applied to the ferromagnetic material, and can be impacted by the AC current flowing through the ferromagnetic material. The magnetic field dependence of the impedance of the ferromagnetic material is controlled by the ability of the magnetization in the ferromagnetic material to respond to the magnetic field generated by the AC current in the ferromagnetic material. Thus, MI sensors that include MI devices employing ferromagnetic materials injected with an AC current will experience a change in impedance as magnetic nanoparticles that have been captured by bioreceptors bound to target analytes of interest pass through a biological area of the MI sensor and apply a magnetic stray field on the ferromagnetic material in the MI device. This change in impedance can be detected and measured to determine the amount of magnetic nanoparticles present, and thus the target analyte of interest.

In aspects disclosed herein, the MI devices include at least one ferromagnetic layer comprised of at least one ferromagnetic material and a conducting layer formed of a conducting material, separated by an insulating layer formed of an insulating material. An exchange bias layer(s) of an anti-ferromagnetic material is directly interfaced to an outer surface of the ferromagnetic layer(s) opposite of the conducting layer. The ferromagnetic material may be a soft, amorphous ferromagnetic material that has a high permeability that is strongly dependent on an external DC magnetic field. This allows the permeability, and thus the skin depth of the ferromagnetic material, to be more easily controlled by a magnetic stray field from magnetic nanoparticles to be detected for a higher GMI ratio and sensitivity. Larger skin depth creates a larger variation in impedance in the presence of an external magnetic field for a given AC current. Providing the conducting layer allows the conducting layer to carry the AC current during sensing to create a magnetic flux in the neighboring ferromagnetic layer(s) to provide a closed magnetic flux loop in the ferromagnetic material layer(s) for maintaining a uniform magnetic field in the ferromagnetic material layer(s). The conducting layer can also enable a larger change in impedance of the ferromagnetic layer(s) to occur in the presence of the magnetic stray field at lower AC current frequencies due to the increase in inductive reactance of the ferromagnetic layer(s) over the resistance of the conducting layer if there is a sufficient difference in resistivity between the conducting and ferromagnetic layer(s). The insulating layer further assists in increasing the GMI ratio and sensitivity by assisting in keeping the AC current confined from leaking and spreading the current density from the conducting layer into the ferromagnetic layer(s). Otherwise, leaked current into the ferromagnetic layer(s) could alter the magnetic field, and thus the magnetic configuration of the ferromagnetic layer(s), thus reducing sensitivity. An exchange bias layer comprising an anti-ferromagnetic material is exchange-coupled to the ferromagnetic layer(s) to pin the interfacial magnetic moments of the ferromagnetic layer(s) to bias the operating point (i.e., from when the external magnetic field is not present) of the MI device for increased sensitivity.

Further, thin film materials can be used to fabricate the MI devices to allow the MI devices to be more easily integrated into an integrated circuit (IC) chip fabricated using semiconductor fabrication methods. For example, an MI device may be fabricated from sputtered materials to form sputtered films according to a sputtering process. This may allow the MI device to be more easily integrated in an IC chip. For example, the MI device could be formed in a back-end-of-line (BEOL) of a complementary metal-oxide semiconductor (CMOS) chip using conventional CMOS BEOL fabrication processes, as opposed to, for example, MI devices that include a coiled core or amorphous wires (e.g., >1 micrometer (μm) in diameter). This would allow the MI device to be easily integrated and interconnected with other circuits of the MI sensor and/or other sensing circuits to provide MI sensors in the CMOS IC chip.

In this regard, in one exemplary aspect, an MI device is provided. The MI device comprises a substrate and an MI structure. The MI structure comprises a conducting layer disposed above the substrate. The conducting layer has a first contact area and a second contact area. The MI structure also comprises an insulating layer disposed above the conducting layer. The MI structure also comprises a ferromagnetic layer disposed above the insulating layer. The ferromagnetic layer comprises a bottom outer surface disposed adjacent to the insulating layer and a top outer surface. The MI structure also comprises an exchange bias layer comprising an anti-ferromagnetic material disposed in contact with the top outer surface of the ferromagnetic layer.

In another exemplary aspect, an MI sensor is provided. The MI sensor comprises an MI device encapsulated in an encapsulation material. The MI device comprises an MI structure. The MI structure comprises a conducting layer disposed above a substrate. The conducting layer has a first contact area and a second contact area. The MI structure also comprises an insulating layer disposed above the conducting layer. The MI structure also comprises a ferromagnetic layer disposed above the insulating layer. The ferromagnetic layer comprises a bottom outer surface disposed adjacent to the insulating layer and a top outer surface. The MI structure also comprises an exchange bias layer comprising an anti-ferromagnetic material disposed in contact with the top outer surface of the ferromagnetic layer. The MI device also comprises a first electrode in electrical contact with the first contact area of the conducting layer, and a second electrode in electrical contact with the second contact area of the conducting layer. The MI sensor also comprises an external channel formed in a void in the encapsulation material. The external channel forms a biological area configured to capture magnetic nanoparticles. The MI sensor also comprises an AC current source circuit electrically coupled to the first contact area and the second contact area of the conducting layer. The AC current source circuit is configured to generate an AC current to flow through the conducting layer. The MI sensor also comprises a sensing circuit. The sensing circuit is configured to receive a sense voltage in response to the magnetic nanoparticles generating a magnetic stray field in the ferromagnetic layer and changing an impedance of the ferromagnetic layer. The sensing circuit is also configured to generate an output voltage based on the sense voltage representing the impedance of the ferromagnetic layer.

In another exemplary aspect, a method of detecting a presence of magnetic nanoparticles in an MI sensor is provided. The method comprises receiving at least one magnetic nanoparticle configured to generate a magnetic stray field bound to a bioreceptor configured to capture a target analyte of interest in at least one external channel in an MI biosensor chip. Each of the at least one external channel forms a biological active area. The MI biosensor chip comprises a plurality of MI devices. Each of the plurality of MI devices comprises a conducting layer disposed above a substrate. The conducting layer has a first contact area and a second contact area, an insulating layer disposed above the conducting layer, and a ferromagnetic layer disposed above the insulating layer. The ferromagnetic layer comprises a bottom outer surface disposed adjacent to the insulating layer and a top outer surface, and an exchange bias layer comprising an anti-ferromagnetic material disposed in contact with the top outer surface of the ferromagnetic layer. The method also comprises generating an AC current to flow through the conducting layer. The method also comprises receiving a sense voltage of the conducting layer in response to the magnetic nanoparticles generating the magnetic stray field in the ferromagnetic layer and changing an impedance of the ferromagnetic layer. The method also comprises generating an output voltage based on the sense voltage representing the impedance of the ferromagnetic layer.

### BRIEF DESCRIPTION OF THE FIGURES

FIG. 1 is a schematic diagram of a biosensing system that employs a biosensor for detecting the presence and/or properties of a biological sample;

FIG. 2 is a schematic diagram of a giant magneto-resistive (GMR) sensor employing a GMR device in a chip whose resistance is configured to change in response to the presence of magnetic nanoparticles, which may be bound to a bioreceptor that is bound to a target analyte of interest, based on a GMR effect;

FIGS. 3A-1 and 3A-2 are front views of an exemplary conductor with different skin depths as a function of respective, different external direct current (DC) magnetic fields (H) applied to the conductor to illustrate a giant magneto-impedance (GMI) effect;

FIGS. 3B-1 and 3B-2 are side views of the exemplary conductor with different skin depths in FIGS. 3A-1 and 3A-2, respectively;

FIG. 4 is an exemplary graph illustrating skin depth of a ferromagnetic material conductor as a function of an external DC magnetic field applied to the ferromagnetic material conductor and the permeability of the ferromagnetic material conductor, wherein an impedance of the conductor is a function of skin depth;

FIG. 5 is an exemplary graph illustrating a change in voltage across a ferromagnetic material conductor as a representation of a change in impedance of the ferromagnetic material conductor, as a function of an external DC magnetic field applied to the ferromagnetic material conductor and an alternating current (AC) current flowing through the ferromagnetic material conductor;

FIG. 6A is an exemplary magneto-impedance (MI) sensor integrated in an integrated circuit (IC) chip and configured to detect the presence of magnetic nanoparticles captured by bioreceptors bound to a target analyte of interest passing through a biological area of the MI sensor, as a function of a magnetic stray field applied by the magnetic nanoparticles on a ferromagnetic material in an MI device embedded in the MI sensor;

FIG. 6B is a top view of the MI sensor in FIG. 6A and further illustrating an exemplary MI device beneath a biological area of the MI sensor, so as to receive a magnetic stray field induced on the MI device by the magnetic nanoparticles passing through the biological area of the MI sensor;

FIG. 7 is a side view of an exemplary MI device for an MI sensor, wherein the MI device comprises film material layers including a ferromagnetic layer, a conducting layer for carrying an AC current during sensing and inducing a magnetic field on the ferromagnetic layer, an insulating layer separating the ferromagnetic layer from the conducting layer for current confinement for improved sensitivity, and an exchange bias layer comprising an anti-ferromagnetic material interfaced to the ferromagnetic layer to pin the interfacial magnetic moments of the ferromagnetic layer to bias the operating point of the MI device for increased sensitivity;

FIG. 8A is an exemplary graph illustrating a shift in MI characteristics of the MI device in FIG. 7 as a result of the exchange bias layer interfaced to the ferromagnetic layer in the MI device, shown as a GMI ratio as a function of an external DC magnetic field induced to the ferromagnetic material conductor and an AC current flowing through the ferromagnetic material conductor;

FIG. 8B is an exemplary graph of B-H curves showing the relationship between magnetization and the magnetic field strength (H) in the ferromagnetic material in the MI device in FIG. 7 with and without the bias exchange layer;

FIG. 9 is a side view of an exemplary complementary metal-oxide semiconductor (CMOS) IC chip that includes an MI sensor that includes an MI device, including but not limited to the MI device in FIG. 7, wherein the MI sensor is fabricated and incorporated in a back-end-of-line (BEOL) of the CMOS IC chip;

FIG. 10 is a schematic diagram of an exemplary MR sensing system that includes an MI sensor employing an MI device, including but not limited to the MI device in FIG. 7, and a sensing circuit configured to generate a voltage signal based on a sensed change in impedance in the MI device of the MI sensor in the presence of magnetic nanoparticles;

FIG. 11 is a flowchart illustrating an exemplary process of the MR sensing system in FIG. 10 for detecting and measuring a presence of magnetic nanoparticles passing through a biological area of the MI sensor;

FIG. 12A is a top view of another exemplary MI sensor in an IC chip, wherein the MI sensor includes a plurality of MI devices disposed beneath a biological area of the MI sensor, so as to receive a magnetic stray field from magnetic nanoparticles passing through the biological area of the MI sensor to detect their presence;

FIG. 12B is a top view of another exemplary MI sensor in an IC chip, wherein the MI sensor includes a single, two-dimensional (2D) MI device disposed beneath a biological area of the MI sensor, so as to receive a magnetic stray field from magnetic nanoparticles passing through the biological area of the MI sensor to detect their presence;

FIG. 13 is a side view of an alternating exemplary MI device that includes a plurality of MI devices that each include a material layer structure of the MI device in FIG. 7 and share a common conducting layer;

FIG. 14 is a top view of another exemplary MI sensor in an IC chip, wherein the MI sensor includes two (2) MI device groups, wherein each MI device group is disposed on adjacent opposite sides of a biological area, and wherein each MI device group includes a plurality of MI devices that can be used in corresponding pairs to provide differential MI sensing for increased sensitivity;

FIG. 15 is a schematic diagram of an exemplary differential MR sensing system that includes an MI sensor employing a plurality of MI devices, including but not limited to the MI devices in FIGS. 7 and 13, and a sensing circuit configured to generate a voltage signal based on a sensed change in impedance in the MI device in the presence of magnetic nanoparticles; and

FIG. 16 is an exemplary biosensor chip that can employ one or more MI sensors employing MI devices, including but not limited to the MI devices in FIGS. 7, 11-14, configured to provide MI sensing of impedance change in response to a presence of magnetic nanoparticles, which may be bound to a bioreceptor that is bound to a target analyte of interest, based on a GMI effect.

### DETAILED DESCRIPTION

With reference now to the drawing figures, several exemplary aspects of the present disclosure are described. The word “exemplary” is used herein to mean “serving as an example, instance, or illustration.” Any aspect described herein as “exemplary” is not necessarily to be construed as preferred or advantageous over other aspects.

Aspects disclosed herein include magneto-impedance (MI) sensors employing current confinement and exchange bias layer(s) for increased MI sensitivity. For example, these MI sensors may be used as biosensors to detect the presence of biological materials. The MI sensing by the MI devices is based on a giant magneto-impedance (GMI) effect. The GMI effect is much more sensitive to a magnetic field than, for example, a giant magneto-resistive (GMR) effect. As an example, a GMI device may be capable of detecting a magnetic stray field down to 10⁻⁸ Oerstead (Oe), to a sensitivity of 100%/Oe. The GMI effect is a change in impedance of a magnetic material resulting from a change in skin depth of the magnetic material as a function of an external direct current (DC) magnetic field applied to the magnetic material and an alternating current (AC) current flowing through the magnetic material. Skin depth is the distance between the surface of a conductor and the point within the conductor where the amplitude of an AC current reduces to a defined percentage (e.g., 37%) of its original value at the surface of the conductor. Skin depth of a conductor is an inverse function of the permeability of the conductor and the frequency of the AC current flowing through the conductor. The permeability of a ferromagnetic material conductor depends on the direction and magnitude of the external magnetic field applied to the ferromagnetic material, and can be impacted by the AC current flowing through the ferromagnetic material. The magnetic field dependence of the impedance of the ferromagnetic material is controlled by the ability of the magnetization in the ferromagnetic material to respond to the magnetic field generated by the AC current in the ferromagnetic material. Thus, MI sensors that include MI devices employing ferromagnetic materials injected with an AC current will experience a change in impedance as magnetic nanoparticles that have been captured by bioreceptors bound to target analytes of interest pass through a biological area of the MI sensor and apply a magnetic stray field on the ferromagnetic material in the MI device. This change in impedance can be detected and measured to determine the amount of magnetic nanoparticles present, and thus the target analyte of interest.

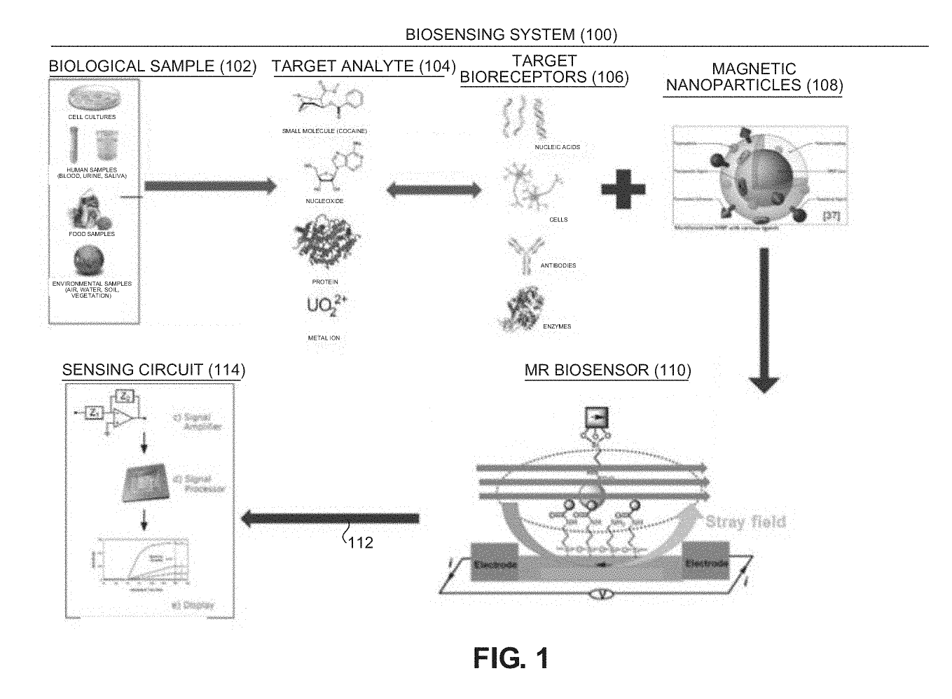

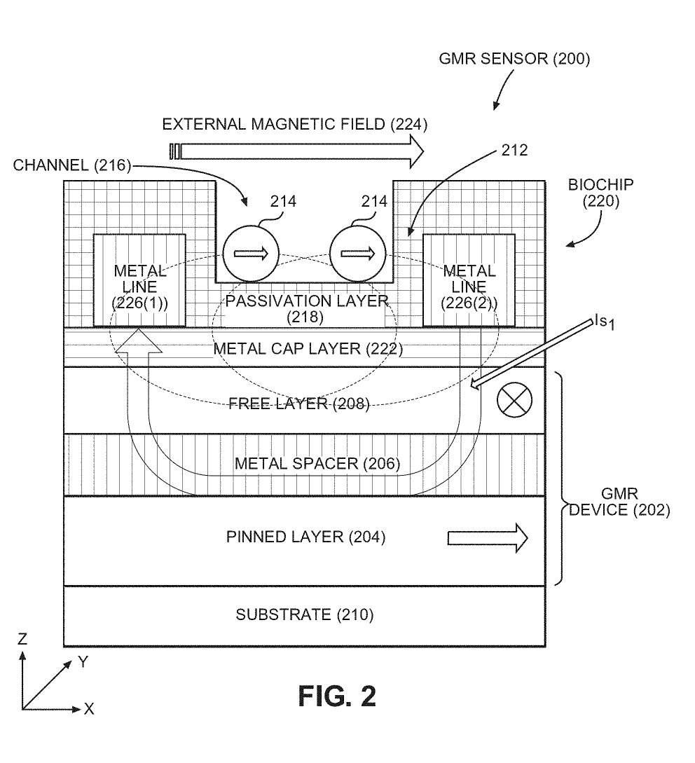

To further illustrate the GMI effect of a magnetic material resulting from a change in skin depth and as a function of an external direct current (DC) magnetic field induced to the magnetic material, FIGS. 3A-1-3B-2 are provided. FIGS. 3A-1 and 3A-2 illustrate front views of a magnetic conductor 300 to show respective differences in skin depths δₘ₁, δₘ₂ as a function of permeability μᵣ of the magnetic conductor 300. As discussed above, the magnetic field dependence of the impedance of a magnetic conductor is controlled by the ability of the magnetization in the magnetic conductor to respond to the magnetic field generated by the AC current flowing in the magnetic conductor 300 based on its permeability. As discussed above, the permeability μᵣ of the magnetic conductor 300 can be changed based on the strength of an externally induced DC magnetic field H_{dc}. FIGS. 3B-1 and 3B-2 illustrate side views of the magnetic conductor 300 in FIGS. 3A-1 and 3A-2, respectively. FIG. 4 is an exemplary graph 400 illustrating skin depths δₘ₁, δₘ₂ of the magnetic conductor 300 as a function of the external DC magnetic field H_{dc} induced by the magnetic conductor 300 and the permeability μᵣ of the magnetic conductor 300.

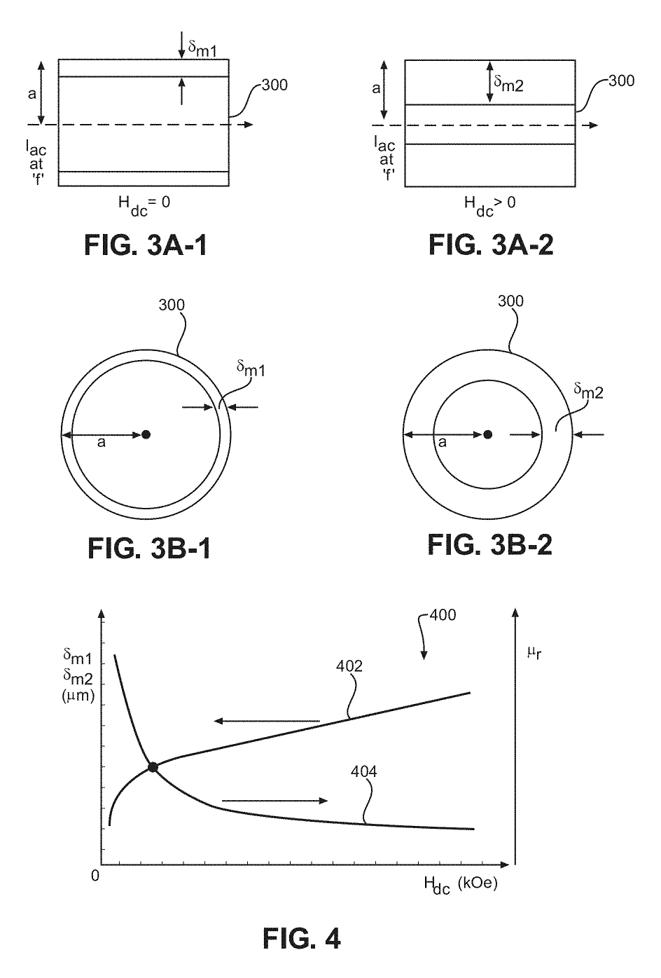

As shown in FIGS. 3A-1 and 3A-2, the magnetic conductor 300 is an elongated circular-shaped wire. For discussion purposes, assume that an AC current I_{ac} is flowing through the magnetic conductor 300 at frequency ‘f.’ The skin depth δₘ of the magnetic conductor 300 is shown as δₘ₁ in FIG. 3A-1 in response to the absence of an external DC magnetic field H_{dc} (i.e., H_{dc}=0). Skin depth δₘ of the magnetic conductor 300 is shown according to the following formula:

$${δ}_{m} = \frac{c}{\sqrt{4 {π}^{2} f {σμ}_{φ}}}$$

wherein:
‘c’ is speed of light;
‘f’ is frequency of an applied AC current; and
μ₀ is the permeability of free space.

However, the skin depth δₘ of the magnetic conductor 300 increases from δₘ₁ to δₘ₂ between FIGS. 3A-1 and 3A-2 in the presence of an external DC magnetic field H_{dc} having an Oe strength greater than 0 (i.e., H_{dc}>0), as shown in curve 402 in the graph 400 in FIG. 4. The skin depth δₘ decreases from δₘ₂ to δₘ₁ between FIGS. 3A-2 and 3A-1 in the presence of the external DC magnetic field R_{dc} as permeability μᵣ decreases as shown in curve 404 in FIG. 4. This is because the permeability μᵣ of the magnetic conductor 300 is dependent on the direction and magnitude of the external DC magnetic field H_{dc} induced by the magnetic conductor 300. The permeability μᵣ of the magnetic conductor 300 experiences a non-linear behavior for a given change in the induced external DC magnetic field H_{dc}, especially for ferromagnetic materials. The strong dependence of permeability μ of a magnetically soft ferromagnetic material to an external DC magnetic field induced to magnetically soft ferromagnetic material in particular gives rise to the GMI effect. As a result of the induced external DC magnetic field H_{dc}, the permeability of the magnetic conductor 300 becomes μᵣ as shown in the curves 402 and 404 in FIG. 4. As a result of the non-linear change in permeability μᵣ of the magnetic conductor 300 in response to the induced external DC magnetic field H_{dc}, the resistance R and the inductance L of the magnetic conductor 300 will experience a large change according to the formulas below.

R=(ρl)/2π(a−δₘ)δₘ
wherein:
   ‘ρ’ is resistivity of the magnetic conductor 300;
   ‘l’ is length of the magnetic conductor 300; and
   ‘a’ is the radius of the magnetic conductor 300.

L=0.175μ₀lf(μᵣ)/ω
wherein:
   ‘μ₀’ is the permeability of free space;
   ‘l’ is length of the magnetic conductor 300; and
   ‘f’ is frequency of an applied AC current I_{ac}; and
   ‘μᵣ’ is the relative permeability of the magnetic conductor 300 with the external DC magnetic field H_{dc} induced.

Impedance Z of the magnetic conductor 300 is as follows:

Z=R+jωL

Thus, impedance Z of the magnetic conductor 300 is an inverse function of skin depth δₘ, because the resistance R and the inductance L of the magnetic conductor 300 are an inverse function of skin depth δₘ. As discussed above, in the presence of the external magnetic field H_{dc}, the initial permeability μ₀ in the magnetic conductor 300 can change significantly thereby causing a significant change in inductance.

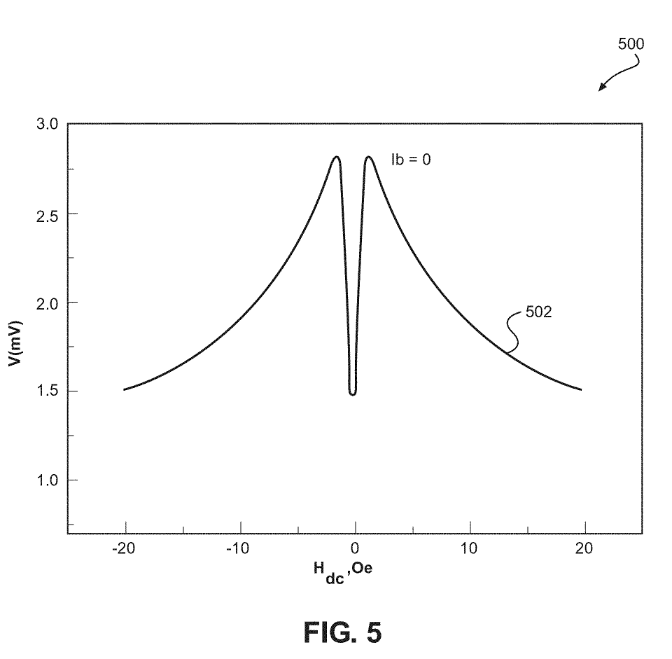

The voltage across a magnetic conductor, such as the magnetic conductor 300 in FIGS. 3A-1-3B-2 is a function of impedance. Thus, a change in voltage across a magnetic conductor, such as the magnetic conductor 300, can be used to determine a change in impedance, and thus the strength of the external DC magnetic field H_{dc} if other variables that affect the GMI effect are known. In this regard, FIG. 5 is an exemplary graph 500 illustrating a change in voltage V across the magnetic conductor 300 as a function of the strength of the external DC magnetic field H_{dc} for an AC current. Curve 502 shows the voltage V response as a function of the external DC magnetic field H_{dc} for a given DC current I_{b} flowing through the magnetic conductor 300.

If, instead of being a cylindrical wire, the magnetic conductor 300 was a thin film ferromagnetic material (FM) sputtered on a non-ferromagnetic material (NM) as a thin layer material stack, the GMI effect would be raised even though the skin effect may be weaker due to the FM material having a reduced skin depth. This is different from the GMI effect in cylindrical wires, such as the magnetic conductor 300 in FIGS. 3A-1-3B-2, because if the sputtered layers are very thin, a higher AC frequency would be needed to have a detectable GMI effect. However, it has been recognized that GMI effect of a thin film structure of a FM/NM is larger and more easily detected with lower AC frequencies injected into the NM material, because of a cross over between the resistance determined by an inner NM conductor versus the inductance related to the outer FM layer as the magnetic field affects the permeability of the FM layer. For example, if the material stack was a FM/NM/FM structure, the GMI effect would be larger and more easily detected with lower AC frequencies injected into the NM material, because of a cross over between the resistance determined by an inner NM conductor versus the inductance related to the outer FM layers as the magnetic field affects the permeability of the FM layers. In this regard, the impedance Z of such a FM/NM/FM structure is follows:

$$Z = {R}_{m} ( 1 - 2 j {μ}_{t} \frac{{d}_{2} {d}_{1}}{{δ}_{1}^{2}} ) , {R}_{m} = l / 2 {bd}_{1} {σ}_{1}$$

wherein:
   d₁=thickness of NM;
   d₂=thickness of FM;
   δ₁=skin depth of NM;
   μₜ=transverse permeability of FM;
   Rₘ=NM resistance;
   b=width of structure; and
   σ₁=conductivity of NM.

Recognizing the GMI effect, an MI sensor can be provided that includes a non-ferromagnetic material in a FN/NM material stack injected with an AC current to undergo changes in skin depth in response to an external DC magnetic field that causes a measurable change in impedance to in turn determine the strength of the external DC magnetic field. In this regard, this MI sensor can be designed as a biosensor that has a biological active area in which magnetic nanoparticles that have been captured by bioreceptors bound to target analytes of interest can pass, and induce a magnetic stray field in the ferromagnetic material in the MI sensor. This change in impedance can be detected and measured to determine the amount of magnetic nanoparticles present, and thus the presence and amount of the target analyte of interest.

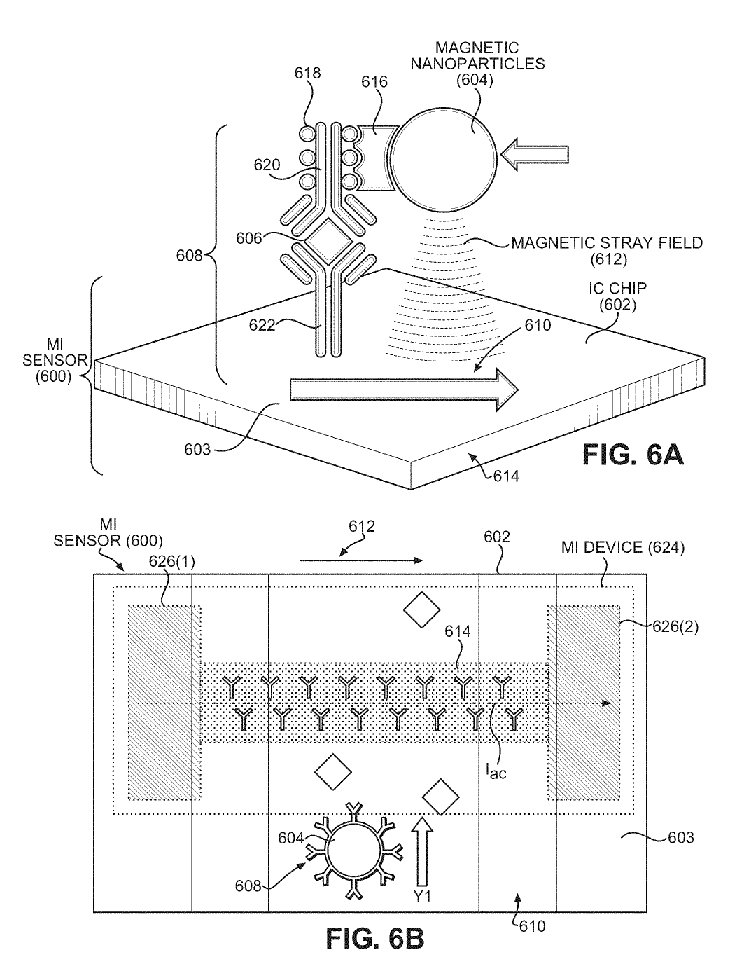

In this regard, FIG. 6A is an exemplary MI sensor 600 integrated in an IC chip 602. A biological area 610 on an outer surface 603 of the IC chip 602 is configured to receive passing magnetic nanoparticles 604 captured by a bioreceptor 606 bound to a target analyte of interest 608. The MI sensor 600 is configured to detect the presence of the magnetic nanoparticles 604 captured by the bioreceptor 606 bound to the target analyte of interest 608 passing through the biological area 610 of the MI sensor 600. The detection of the magnetic nanoparticles 604 is a function of a magnetic stray field 612 induced by the magnetic nanoparticles 604 in a ferromagnetic material 614 embedded in the MI sensor 600, which is shown in a top view of the MI sensor 600 in FIG. 6B. For example, the target analyte of interest 608 may be avidin 616, a biotin 618, a biotinylated antibody 620, or an immobilized antibody 622, as examples. FIG. 6B is a top view of the MI sensor 600 in FIG. 6A and further illustrates an exemplary MI device 624 located beneath the biological area 610 of the MI sensor 600, which is in the form of an external channel in this example. The external channel may be formed by an external void in the IC chip 602. The MI sensor 600 is provided as the IC chip 602 that has been encapsulated to embed the MI device 624 in the IC chip 602. The MI device 624 is arranged to be located underneath the biological area 610 so that as the magnetic nanoparticles 604 pass through the biological area 610 shown by direction Y₁, as shown in hidden lines in FIG. 6B. The magnetic stray field 612 from the magnetic nanoparticles 604 passing through the biological area 610 is induced in the ferromagnetic material 614 of the MI device 624. The MI device 624 includes the ferromagnetic material 614 in electrical contact between two electrodes 626(1), 626(2). As will be discussed in more detail below, an AC current I_{ac} can be directed to flow between the electrodes 626(1), 626(2) to cause the ferromagnetic material 614 to have a skin depth that can then be controlled by the magnetic stray field 612 induced in the ferromagnetic material 614 as a result of the magnetic nanoparticles 604 passing through the biological area 610 to cause a change in impedance of the ferromagnetic material 614 for the GMI effect, as previously discussed above.

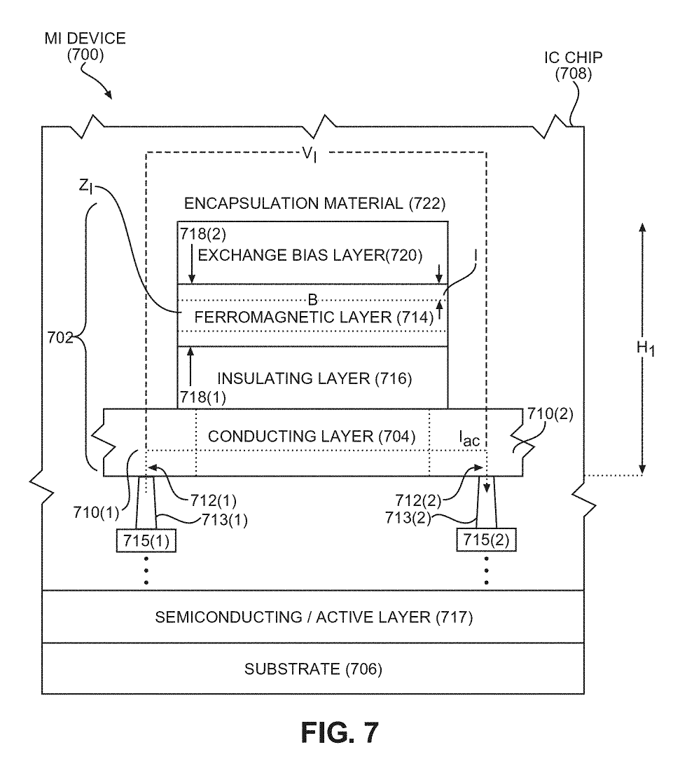

FIG. 7 is a side view of an exemplary MI device 700 that can be used as the MI device 624 in the MI sensor 600 in FIGS. 6A and 6B. The MI device 700 includes an MI structure 702 that includes a conducting layer 704 disposed above a substrate 706. For example, the MI structure 702 in FIG. 7 is included in an IC chip 708 that includes the substrate 706 and may include other electronic circuits and components. The conducting layer 704 is comprised of one or more conducting materials, which can include metal materials including but not limited to Copper (Cu), Silver (Ag), Gold (Au), or other metal material alloys. First and second electrodes 710(1), 710(2) of a conductive material are also formed above the substrate 706 and in electrical contact with a first contact area 712(1) and a second contact area 712(2), respectively, of the conducting layer 704. The first and second contact areas 712(1), 712(2) of the conducting layer 704 in this example are electrically coupled to vertical interconnect accesses (vias) 713(1), 713(2) respectively. The vias 713(1), 713(2) may be electrically coupled to metal lines 715(1), 715(2) to provide interconnectivity with a device, such as a transistor, in a semiconducting/active layer 717 in the IC chip 708, to supply an AC current I_{ac} to flow through the conducting layer 704 as shown in FIG. 7.

With continuing reference to FIG. 7, a magnetic flux is generated in a ferromagnetic layer 714 in response to an AC current I_{ac} flowing through the conducting layer 704. To generate this magnetic flux, the AC current I_{ac} is injected to flow between the electrodes 710(1), 710(2) so that the AC current I_{ac} flows into the conducting layer 704 between the first contact area 712(1) to the second contact area 712(2) of the conducting layer 704. As a result, the ferromagnetic layer 714 provided in the MI device 700 above the conducting layer 704 will have a skin depth that is a function of the permeability of the ferromagnetic layer 714 and the frequency of the AC current I_{ac}, as previously described. The skin depth of the ferromagnetic layer 714 can be controlled by a magnetic stray field induced in the ferromagnetic layer 714 to cause a change in impedance Z₁ of the ferromagnetic layer 714 according to a GMI effect. The change in impedance Z₁ can be sensed through a change in voltage V₁ across the conducting layer 704. In this manner, a change in impedance Z₁ of the ferromagnetic layer 714 as a result of the magnetic stray field inducted in the ferromagnetic layer 714 by the magnetic nanoparticles 604 shown in FIG. 6A for example, can be detected by the change in voltage V₁ across the conducting layer 704.

Providing the conducting layer 704 separately from the ferromagnetic layer 714 allows the AC current I_{ac} to be carried in the conducting layer 704 to create the magnetic flux during sensing to create a magnetic flux in the ferromagnetic layer 714 to provide a closed magnetic flux loop in the ferromagnetic layer 714. This assists in maintaining a uniform magnetic field in the ferromagnetic layer 714. Providing the conducting layer 704 separate from the ferromagnetic layer 714 to carry AC current I_{ac} can also enable a larger change in impedance of the ferromagnetic layer 714 to occur in the presence of a magnetic stray field at lower AC current I_{ac} frequencies. This is due to the increase in inductive reactance of the ferromagnetic layer 714 over the resistance of the conducting layer 704 if there is a sufficient difference in resistivity between the conducting layer 704 and ferromagnetic layer 714. The skin effect causes the effective resistance R of the ferromagnetic layer 714 to increase at higher frequencies where the skin depth is smaller, thus reducing the effective cross-section of the ferromagnetic layer 714.

In this example, the ferromagnetic layer 714 is comprised of one or more ferromagnetic materials. In one example, the ferromagnetic material of the ferromagnetic layer 714 is a soft, amorphous ferromagnetic material, examples of which include Cobalt (Co) Silicon (Si) Boron (B) (CoSiB), Co Iron (Fe) SiB (CoFeSiB), Nickel (Ni) Fe (NiFe), CoFeB, Co Fe Vanadium (V) B (CoFeVB), and CoFeSi Noobium (Nb) Copper (Cu) B (CoFeSiNbCuB). Soft, amorphous ferromagnetic materials exhibit excellent GMI response due to their very soft magnetic properties and low magnetostriction, meaning their magnetization varies significantly in the presence of a smaller applied external magnetic field H. Thus, it may be desired to provide for the ferromagnetic layer 714 in the MI device 700 in FIG. 7 to be of a soft amorphous magnetic material and to have a lower anisotropy field. This allows the permeability, and thus the skin depth of the ferromagnetic layer 714, to be more easily controlled by a magnetic stray field from magnetic nanoparticles to be detected for a higher GMI ratio and sensitivity. Larger skin depth creates a larger variation in impedance Z of the ferromagnetic layer 714 in the presence of an external magnetic field for a given AC current.

With continuing reference to FIG. 7, an insulating layer 716 of one or more insulating materials is formed between the ferromagnetic layer 714 and the conducting layer 704 in the MI device 700 in this example. A bottom outer surface 718(1) of the ferromagnetic layer 714 is disposed adjacent to the insulating layer 716. The insulating layer 716 further assists in increasing the GMI ratio and sensitivity of the MI device 700 by assisting in keeping or confining the AC current I_{ac} from leaking and spreading the current density from the conducting layer 704 into the ferromagnetic layer 714. Otherwise, leaked AC current I_{ac} into the ferromagnetic layer 714 could alter the magnetic field B therein, and thus the magnetic configuration of the ferromagnetic layer 714, thus reducing its sensitivity. Non-limiting examples of insulating materials that may be employed in the insulating layer 716 include Silicon Oxide (SiO₂), Hafnium Oxide (HfOx), Magnesium Oxide (MgO), and Aluminum Oxide (AlOx).

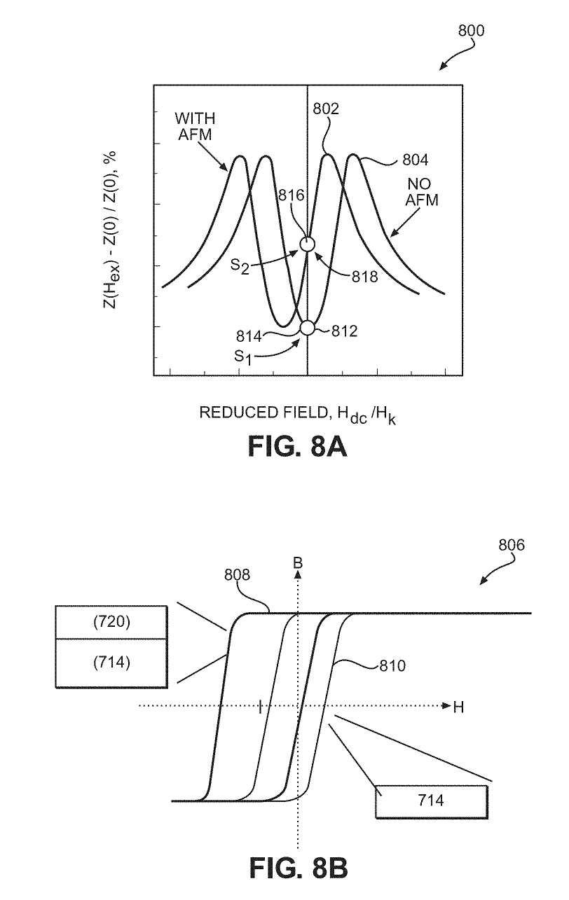

With continuing reference to FIG. 7, the MI device 700 also includes an exchange bias layer 720 that is comprised of an anti-ferromagnetic material. For example, the exchange bias layer 720 could be a material layer of Iridium (Ir) Manganese (Mn) (IrMn), Platinum (Pt) Mn (PtMn), Nickel Oxide (NiO), and Cobalt O (CoO) as non-limiting examples. The exchange bias layer 720 is disposed in contact with a top outer surface 718(2) of the ferromagnetic layer 714. The exchange bias layer 720 is exchange-coupled to the ferromagnetic layer 714 to pin the interfacial magnetic moments of the ferromagnetic layer 714 to bias the operating point (i.e., from when the external magnetic field H is not present) of the MI device 700 for increased sensitivity. This is shown by example in the graphs in FIGS. 8A and 8B. FIG. 8A is an exemplary graph 800 illustrating the shift of the MI characteristics of the MI device 700 in FIG. 7 as a result of the exchange bias layer 720 interfaced to the ferromagnetic layer 714. The graph 800 in FIG. 8A illustrates curves 802, 804 of GMI ratio (i.e., (Z(H_{dc})−Z(0))/Z(0)) and sensitivity (i.e., d(ΔZ/Z₀)/dH_{dc}×100%) of the MI device 700 as a function of reduced magnetic field (H_{dc}/Hₖ), with (curve 802) and without (curve 804) the exchange bias layer 720 shown in FIG. 7 provided, respectively, which is this case is an AFM exchange bias layer 720. ‘Z’ is impedance, ‘H_{dc}’ is an external applied DC magnetic field, and ‘Hₖ, ’ is an anisotropy field of the ferromagnetic layer 714. FIG. 8B is a graph 806 of exemplary B-H curves 808, 810 showing the relationship between magnetic flux density and the magnetic field H strength in the ferromagnetic layer 714 in the MI device 700 in FIG. 7 with and without the exchange bias layer 720, respectively.

Note that in FIG. 8A, for an initial operating point 812 of the MI device 700 in an area 814 around a zero external DC magnetic field H_{dc}, meaning that no external DC magnetic field H_{dc} is applied, the slope S₁ is smaller for a change in the external DC magnetic field H_{dc} than for an initial operating point 816 at zero external DC magnetic field H_{dc} caused by biasing the magnetic moments of the ferromagnetic layer 714. The slope S₂ is larger in an area 818 around the initial operating point 816 for a change in the external DC magnetic field H_{dc} because of the biasing of interfacial magnetic moments of the ferromagnetic layer 714 by the exchange bias layer 720, than in the area 814 around the initial operating point 812 when the ferromagnetic layer 714 is not biased by the exchange bias layer 720. Thus, the MI device 700 with the exchange bias layer 720 causes the ferromagnetic layer 714 to experience a larger change in impedance in response to the external DC magnetic field H_{dc} than otherwise with the exchange bias layer 720 for improved performance and sensitivity. The exchange bias layer 720 also avoids the need to provide a separate external magnetic field, such as from an external coil or permanent magnet, to bias the MI device 700. It would be more difficult and consume more area to provide for such a coil in the IC chip 708 in FIG. 7. It may also consume additional power to produce a separate external magnetic field with a coil in an undesired manner.

With continuing reference to FIG. 7, the MI device 700 is encapsulated with an encapsulation material 722 as part of the IC chip 708. Examples of the encapsulation material 722 include, but are not limited to, Silicon Oxide (SiO₂) and Silicon Nitride (SiN). Further, the material layers in the MI device 700 in FIG. 7 can be fabricated as film materials, including thin film materials, to allow the MI device 700 to be more easily integrated into an IC chip 708 fabricated using semiconductor fabrication methods. For example, the layers in the MI device 700 may be fabricated from sputtered film materials according to a sputtering process. Fabricating the layers in the MI device 700 as thin films allows the overall size of the MI device 700 to be reduced and thereby reduces the distance from the detected analyte particle to the MI device 700, which improves sensitivity, reduces power and allows the MI device 700 to be more easily scaled down with potential cost reduction. For example, with reference to FIG. 7, the conducting layer 704 may be fabricated or sputtered as a thin film to have a thickness of approximately between 200-500 nanometers (nm). The insulating layer 716 may be fabricated or sputtered as a thin film to have a thickness of approximately between 10-20 nm. The ferromagnetic layer 714 may be fabricated or sputtered as a thin film to have a thickness of approximately between 100-200 nm. The exchange bias layer 720 may be fabricated or sputtered as a thin film to have a thickness of approximately between 5-25 nm. The entire height H₁ of the MI device 700 may be fabricated to be two (2) micrometers (μm) or less.

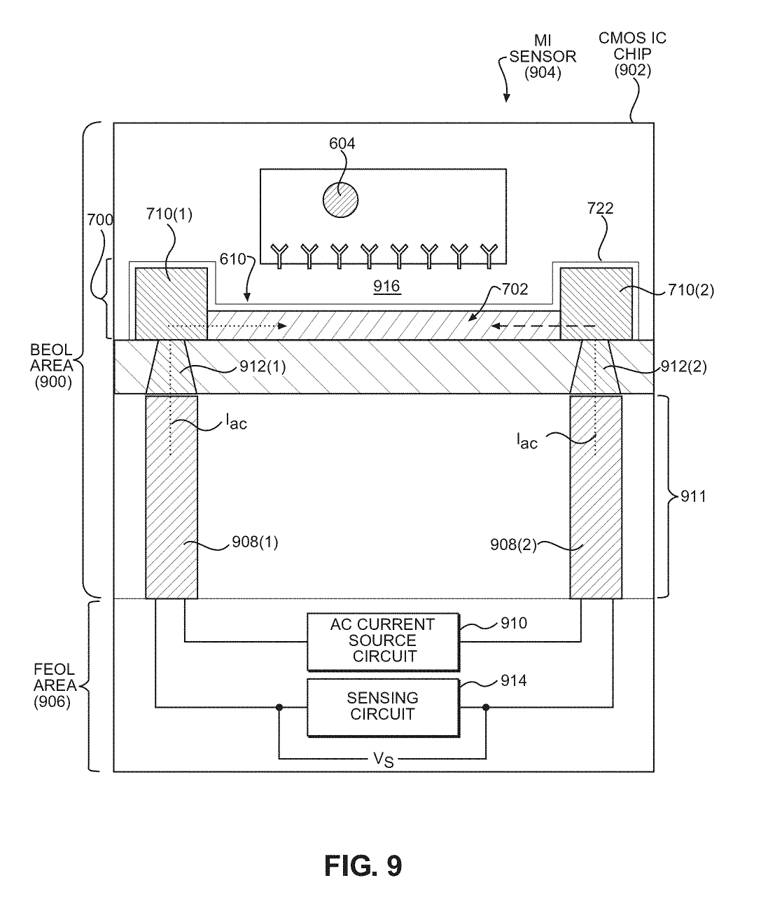

Further, the MI device 700 in FIG. 7 could be formed in a BEOL area 900 of a CMOS IC chip 902 as shown in FIG. 9 for example to provide an MI sensor 904. FIG. 9 illustrates a side view of the exemplary CMOS IC chip 902 that includes the MI device 700 in FIG. 7. The CMOS IC chip 902 includes a front-end-of-line (FEOL) area 906 where active CMOS devices can be provided. For example, an AC current source circuit 910 may be included in the FEOL area 906 and electrically coupled to the first and second electrodes 710(1), 710(2) through metal lines 908(1), 908(2) in one or more metal layer(s) 911 and vertical interconnect accesses (VIAs) 912(1), 912(2) to inject the AC current I_{ac} through the conducting layer 704 of the MI device 700 (see FIG. 7). Further, the FEOL area 906 may also include a sensing circuit 914 that is also electrically coupled to the first and second electrodes 710(1), 710(2) through the metal lines 908(1), 908(2) in the one or more metal layer(s) 911 and VIAs 912(1), 912(2) to sense the impedance in the ferromagnetic layer 714 as a function of voltage V. For example, the sensing circuit 914 may be configured to sense a sense voltage Vs in the ferromagnetic layer 714 in response to the magnetic nanoparticles generating a magnetic stray field in the ferromagnetic layer 714 and changing the impedance of the MI device 700. The sensing circuit 914 may be configured to generate an output voltage Vₒ based on the sense voltage Vₛ representing the impedance of the MI device 700 in response to an external magnetic field induced in the MI device 700 from magnetic nanoparticles passing in an external channel 916 formed in a void of the encapsulation material 722.

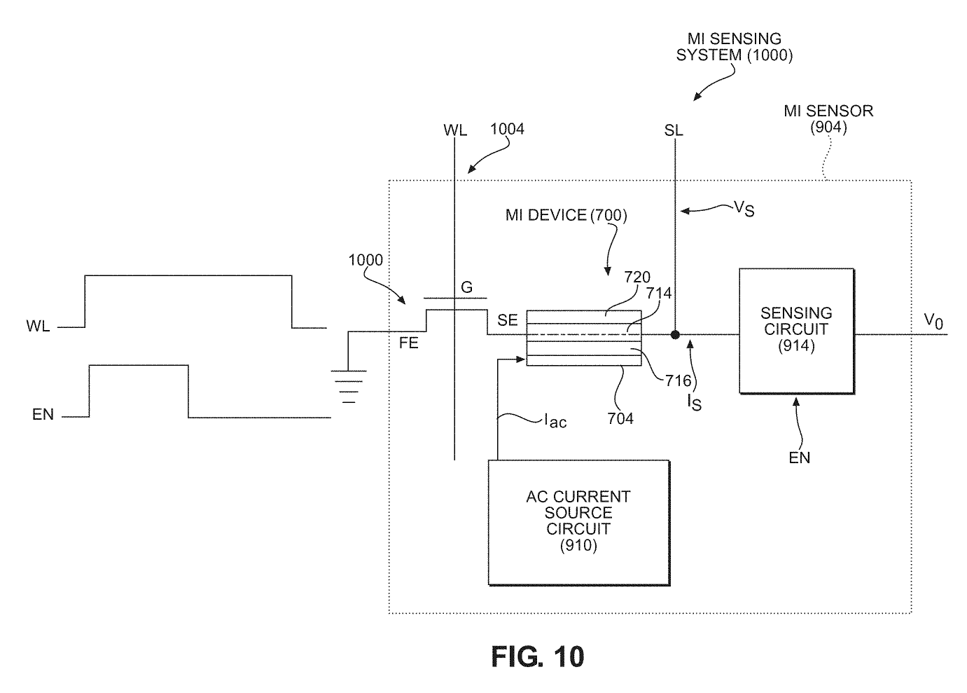

FIG. 10 is a schematic diagram of an exemplary MI sensing system 1000 that can include the MI sensor 904 in FIG. 9 employing the MI device 700, to generate an output voltage Vₒ based on the sensed change in impedance in the MI device 700 in the presence of magnetic nanoparticles. In this regard, the MI device 700 of the MI sensor 904 is shown coupled to an access transistor 1002 to control the connection of the MI device 700 to the sensing circuit 914. The access transistor 1002 includes a gate (G), a first electrode (FE), and a second electrode (SE). The gate (G) is coupled to a word line (WL). The second electrode (SE) is electrically coupled to the ferromagnetic layer 714. The ferromagnetic layer 714 is also coupled to a source line (SL). The ferromagnetic layer 714 is configured to receive the sense voltage Vₛ based on the impedance of the ferromagnetic layer 714 in response to a control signal 1004 on the word line (WL) activating the access transistor 1002 and the source line (SL).

With continuing reference to FIG. 10, the sensing circuit 914 is configured to receive the sense voltage Vₛ from the MI device 700 of the MI sensor 904 in response to an enable signal EN indicating an enable state (high state in this example). In response, the sensing circuit 914 is configured to generate an output voltage Vₒ based on the sense voltage Vₛ representing the impedance of the MI device 700.

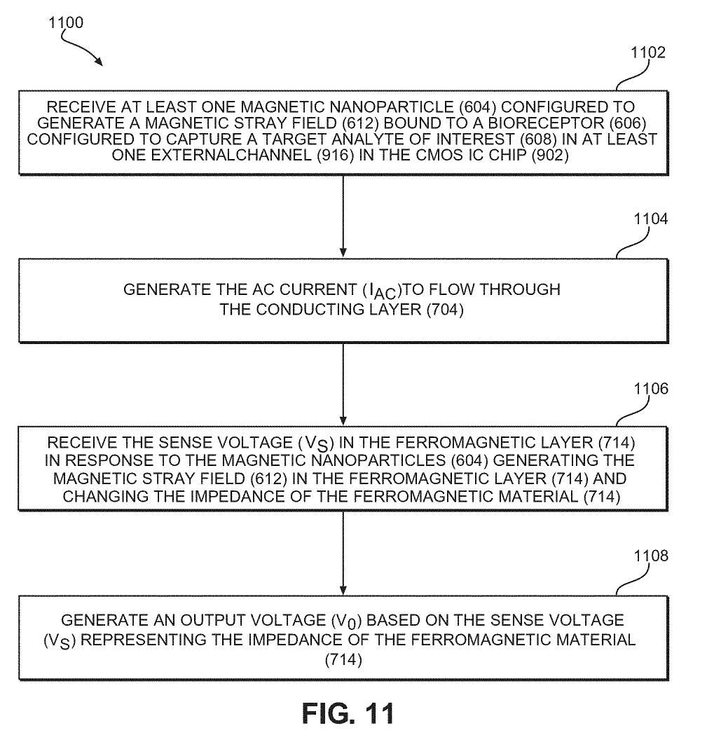

FIG. 11 is a flowchart illustrating an exemplary process 1100 of the MI sensing system 1000 in FIG. 10 for detecting and measuring a presence of magnetic nanoparticles passing through the biological area of the MI sensor 904 in FIG. 9. In this regard, the MI sensor 904 receives at least one magnetic nanoparticle 604 configured to generate a magnetic stray field 612 bound to a bioreceptor 606 configured to capture a target analyte of interest 608 in at least one external channel 916 in the CMOS IC chip 902 (block 1102). The at least one external channel 916 forms a biological active area. The process 1100 also includes the AC current source circuit 910 generating the AC current I_{ac} to flow through the conducting layer 704 (block 1104). The process 1100 also includes the sensing circuit 914 receiving the sense voltage Vₛ in the ferromagnetic layer 714 in response to the magnetic nanoparticles 604 generating the magnetic stray field 612 in the ferromagnetic layer 714 and changing the impedance of the ferromagnetic layer 714 (block 1106). The sensing circuit 914 generates an output voltage Vₒ based on the sense voltage Vₛ representing the impedance of the ferromagnetic layer 714 (block 1108).

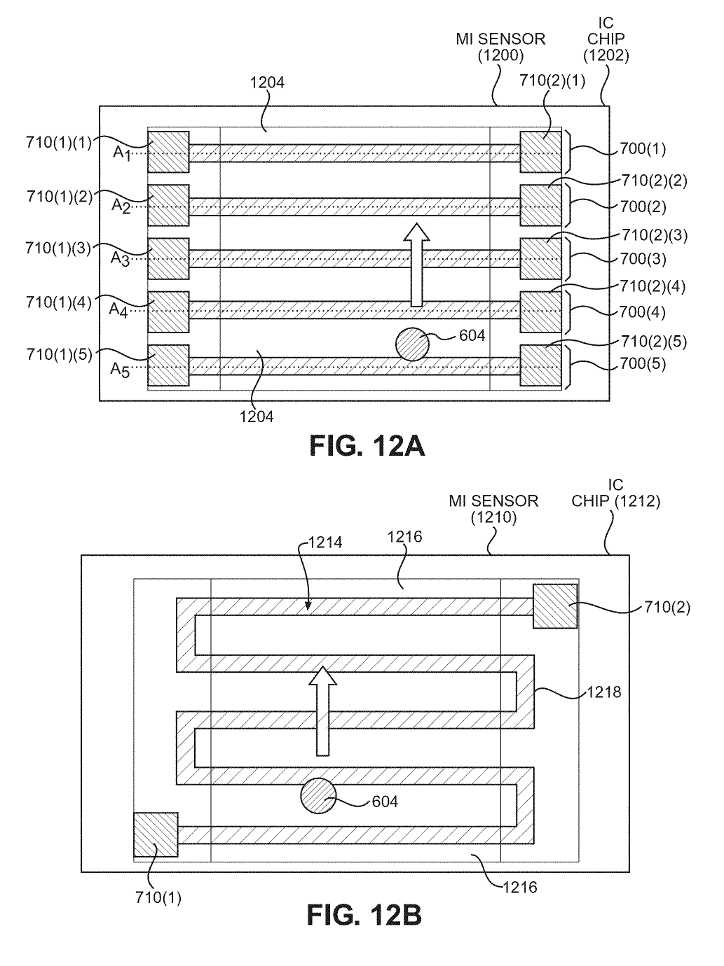

MI devices, like the MI device 700 in FIG. 7, can be provided in an IC chip in a number of different manners and arrangements. For example, FIG. 12A is a top view of another exemplary MI sensor 1200 in an IC chip 1202, wherein the MI sensor 1200 includes a plurality of MI devices 700(1)-700(5) disposed beneath an external channel 1204 to form a biological area. The material layers of the MI devices 700(1)-700(5) are as provided in the MI device 700 in FIG. 7 in this example, and thus will not be re-described. The MI devices 700(1)-700(5) in the MI sensor 1200 are each aligned along longitudinal axes A₁-A₅ substantially parallel with each other. As the magnetic nanoparticles 604 pass through the external channel 1204, the magnetic stray field from the magnetic nanoparticles 604 is induced in the ferromagnetic layer 714 of the respective MI devices 700(1)-700(5). Separate dedicated AC current source circuits and sensing circuits like the AC current source circuit 910 and the sensing circuit 914 in FIG. 9 may be included in the MI sensor 1200 for each MI device 700(1)-700(5). Alternatingly, a shared AC current source circuit and sensing circuit like the AC current source circuit 910 and sensing circuit 914 in FIG. 9 may be provided in the MI sensor 1200 for all the MI devices 700(1)-700(5). The generation of the AC current I_{ac} and the sensing of the sense voltage Vₛ (see FIG. 9) may be multiplexed between the shared AC current source circuit and sensing circuit.

FIG. 12B is a top view of another exemplary MI sensor 1210 in an IC chip 1212, wherein the MI sensor 1210 includes a single, two-dimensional (2D) MI device 1214 disposed beneath an external channel 1216 to form a biological area. The material layers of the 2D MI device 1214 are as provided in the MI device 700 in FIG. 7 in this example, and thus will not be re-described. The MI device 1214 has a serpentine MI structure 1218 between and electrically contacting the first and second electrodes 710(1), 710(2). This structure provides for the MI structure 1218 to be located underneath a larger area of the external channel 1216 with one MI device 1214. An AC current source circuit and sensing circuit like the AC current source circuit 910 and sensing circuit 914 in FIG. 9 may be provided in the MI sensor 1210 for the MI device 1214.

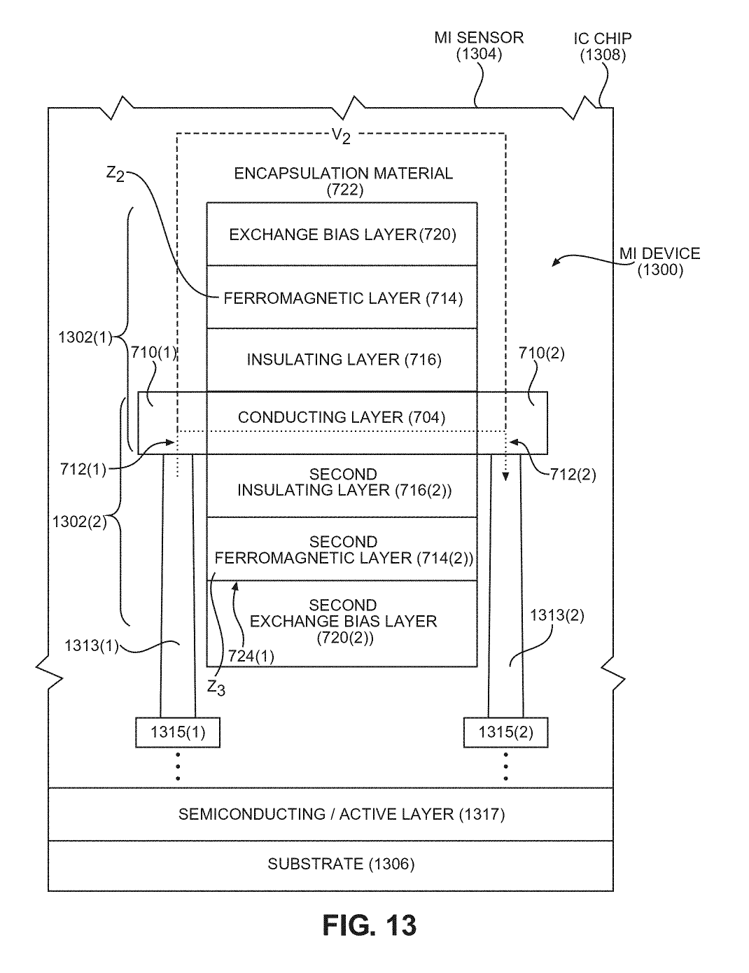

Other structures can be provided that include an insulating layer and exchange bias layer for an MI device similar to the MI device 700 in FIG. 7. For example, FIG. 13 is a side view of an alternative exemplary MI device 1300 for an MI sensor 1304 that includes a FM/NM/FM stack structure. As previously discussed above, the GMI effect would be larger and more easily detected in a FM/NM/FM structure, with lower AC frequencies injected into the NM material. This is because of a cross over between the resistance determined by an inner NM conductor versus the inductance related to the outer FM layers as the magnetic field affects the permeability of the FM layers. In this regard, the MI device 1300 in FIG. 13 includes first and second MI structures 1302(1), 1302(2) that each include a material layer structure similar to the MI structure 702 in FIG. 7. Common components and material layers between the MI structure 1302(1) in FIG. 13 and the MI structure 702 in FIG. 7 are shown with common element numbers and will not be re-described. The first and second contact areas 712(1), 712(2) of the conducting layer 704 in this example are electrically coupled to vias 1313(1), 1313(2), respectively. The vias 1313(1), 1313(2) may be electrically coupled to metal lines 1315(1), 1315(2) to provide interconnectivity with a device, such as a transistor, in a semiconducting/active layer 1317 on a substrate 1306 in the IC chip 1308, to supply the AC current I_{ac} to flow through the conducting layer 704 as shown in FIG. 13.

In the example of the MI device 1300 shown in FIG. 13, the conducting layer 704 is shared between both MI structures 1302(1), 1302(2). The second MI structure 1302(2) in FIG. 13 includes the conducting layer 704 disposed above a second insulating layer 716(2). The previous discussion regarding the insulating layer 716 in FIG. 7 is also applicable to the second insulating layer 716(2) in the second MI structure 1302(2). The second MI structure 1302(2) also includes a second ferromagnetic layer 714(2) disposed below the second insulating layer 716(2). The previous discussion regarding the ferromagnetic layer 714 in FIG. 7 is also applicable to the second ferromagnetic layer 714(2) in the second MI structure 1302(2). The second MI structure 1302(2) also includes a second exchange bias layer 720(2) directly contacting a bottom outer surface 724(1) of the second ferromagnetic layer 714(2) to provide an exchange coupling. The previous discussion regarding the exchange bias layer 720 in FIG. 7 is also applicable to the second exchange bias layer 720(2) in the second MI structure 1302(2). The complex impedances Z₂, Z₃ in the ferromagnetic layers 714, 714(2) is configured to increase an overall impedance of the MI device 1300 in response to an external induced magnetic field with the AC current I_{ac} injected into the conducting layer 704. This change in impedances Z₂ and Z₃ can be sensed through a change in voltage V₂ across the conducting layer 704. In this manner, a change in impedances Z₂, Z₃ in the ferromagnetic layers 714, 714(2) as a result of the magnetic stray field inducted in the ferromagnetic layers 714, 714(2) by the magnetic nanoparticles 604 shown in FIG. 6 for example, can be detected by the change in voltage V₂ across the conducting layer 704.

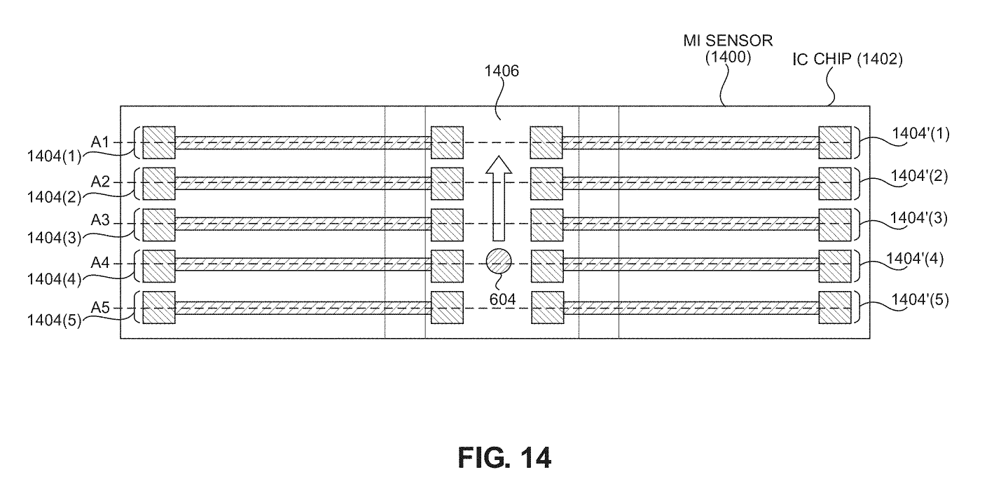

A differential sensing method employing MI devices like the MI devices 700, 1300 in FIGS. 7 and 13 can also be implemented between separate paired MI devices. In this regard, FIG. 14 is a top view of another exemplary MI sensor 1400 in an IC chip 1402 that includes a plurality of MI devices 1404(1)-1404(5), 1404′(1)-1404′(5) disposed beneath an external channel 1406 to form a biological area. Each, any, or all of the MI devices 1404(1)-1404(5), 1404′(1)-1404′(5) can be the MI devices 700, 1300 in FIGS. 7 and 13 as non-limiting examples. The MI devices 1404(1)-1404(5) are arranged like those described above in FIG. 12A and thus will not be re-described. The MI sensor 1400 also includes complementary MI devices 1404′(1)-1404′(5) disposed along the respective longitudinal axes A₁-A₅. The respective MI devices 1404(1) and 1404′(1) form a differential pair of MI devices. The respective MI devices 1404(2) and 1404′(2) form another differential pair of MI devices, and so on. The external channel 1406 is disposed between the MI devices 1404(1)-1404(5), 1404′(1)-1404′(5) as shown in FIG. 14. Thus, as the magnetic nanoparticles 604 pass through the external channel 1406, the external DC magnetic field is induced on the respective pairs of MI devices 1404(1) and 1404′(1), 1404(2) and 1404′(2), 1404(3) and 1404′(3), 1404(4) and 1404′(4), and 1404(5) and 1404′(5).

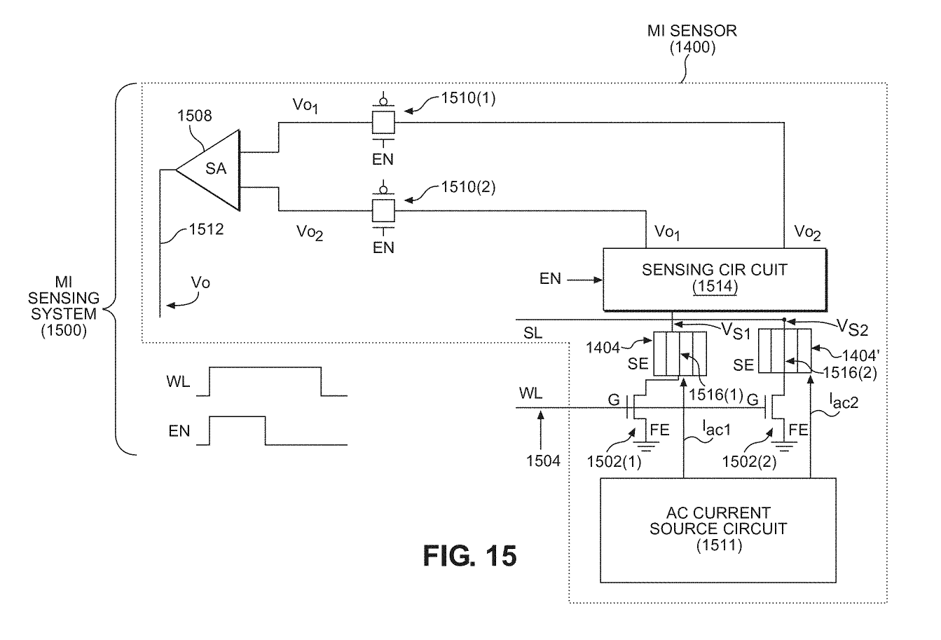

FIG. 15 is a schematic diagram of an exemplary MI sensing system 1500 that can include the MI sensor 1400 in FIG. 14 and respective pairs of MI devices 1404(1)-1404(5), 1404′(1)-1404′(5) to generate an output voltage Vₒ based on the sensed differential change in impedance in a respective pair of MI devices 1404(1) and 1404′(1), 1404(2) and 1404′(2), 1404(3) and 1404′(3), 1404(4) and 1404′(4), and 1404(5) and 1404′(5). FIG. 15 shows a pair of MI devices 1404, 1404′. In this regard, an AC current source circuit 1511 is provided that is configured to generate the AC current l_{ac} to flow through conducting layers 1516(1), 1516(2) during sensing operations. Respective ferromagnetic layers of the MI devices 1404, 1404′ of the MI sensor 1400 are shown coupled to respective access transistors 1502(1), 1502(2) to control establishing a circuit in the MI devices 1404, 1404′ for first and second sense voltages Vₛ₁ and Vₛ₂. The access transistors 1502(1), 1502(2) each include a gate (G), a first electrode (FE), and a second electrode (SE). The gate (G) is coupled to a word line (WL). The second electrode (SE) of the access transistor 1502(1) is electrically coupled to the ferromagnetic layer of the MI device 1404. The second electrode (SE) of the access transistor 1502(2) is electrically coupled to the ferromagnetic layer of the MI device 1404′. The ferromagnetic layers of the MI devices 1404, 1404′ are also coupled to a source line (SL). The ferromagnetic layers are configured to receive the sense voltages Vₛ₁ and Vₛ₂ based on the impedance of the ferromagnetic layers in the MI devices 1404, 1404′ in response to a control signal 1504 on the word line (WL) activating the access transistors 1502(1), 1502(2) and a sense voltage Vₛ applied to the source line (SL).

With continuing reference to FIG. 15, a sensing circuit 1514 is provided and configured to receive the sense voltages Vₛ₁ and Vₛ₂ from the MI devices 1404, 1404′ of the MI sensor 1400 in response to an enable signal EN indicating an enable state (high state in this example). In response, the sensing circuit 1514 is configured to generate output voltages Vₒ₁ and Vₒ₂ based on the sense voltages Vₛ₁ and Vₛ₂ representing the impedance of the MI devices 1404, 1404′.

With continuing reference to FIG. 15, a sense amplifier (SA) 1508 is also provided in the MI sensing system 1500. The sense amplifier 1508 is configured to receive the first and second sensed output voltages Vₒ₁ and Vₒ₂ from the sensing circuit 1514. In this example, a first input circuit 1510(1) and a second input circuit 1510(2) are provided in the form of pass gates to control the timing of the sense amplifier 1508 receiving the first and second sensed output voltages Vₒ₁ and Vₒ₂ from the sensing circuit 1514 based on the enable signal EN. The first input circuit 1510(1) is configured to pass the first sensed output voltages Vₒ₁ and the second input circuit 1510(2) is configured to pass the second sensed output voltage Vₒ₂ during a second sensing phase SS2. The sense amplifier 1508 is configured to sense the first and second sensed output voltages Vₒ₁ and Vₒ₂ based on the differential voltage therebetween to generate an amplified differential output voltage Vₒ on an output node 1512 indicative of the impedances of the ferromagnetic layers of the MI devices 1404, 1404′.

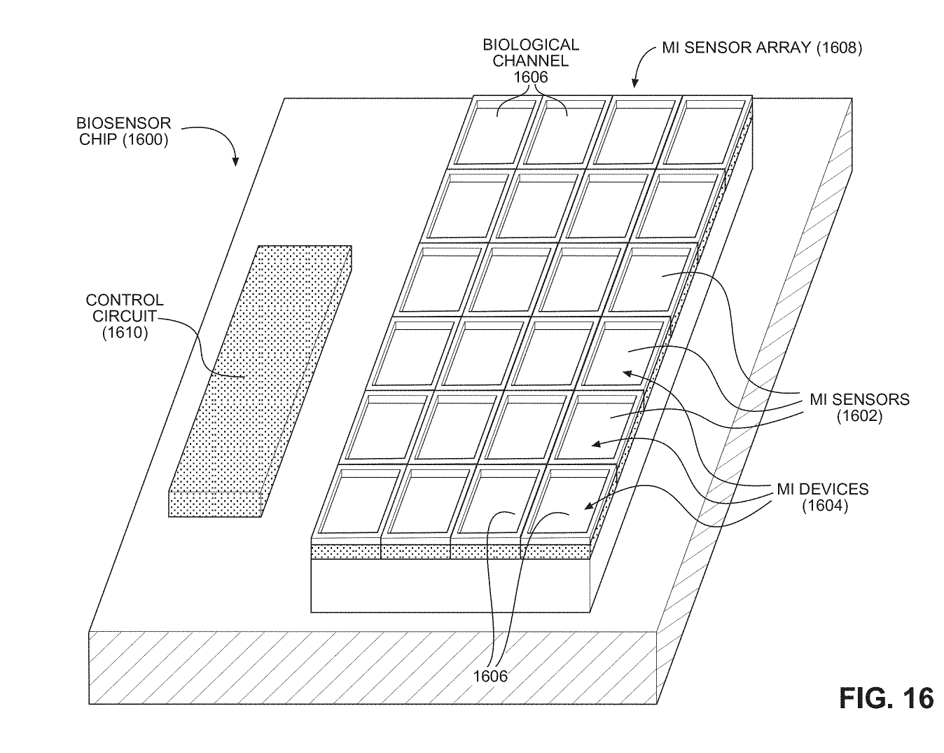

FIG. 16 is an exemplary biosensor chip 1600 that can employ one or more MI sensors 1602 that include one or more ferromagnetic layers and a conducting layer that carries an AC current separated by an insulating layer, such as the MI sensors 600, 904, 1200, 1210, 1304, 1400 in FIGS. 6, 9, 12A, 12B, 13, and 14, as examples. The biosensor chip 1600 may be provided in different applications, including wearable devices, point-of-care devices for point-of-care applications, a bacteria inflection diagnostics device for bacterial infection detection applications, a cancer detection device for cancer detection, a heart disease diagnostic device for detecting heart disease, a food safety monitoring device for food monitoring applications, etc. The magnetic nanoparticles may be bound to a bioreceptor that is bound to a target analyte of interest, in a biological channel 1606 disposed in the MI sensors 1602 and above MI devices 1604, based on a GMI effect. As shown in FIG. 16, the biosensor chip 1600 may have an MI sensor array 1608 that contains the plurality of MI sensors 1602. A control circuit 1610 may also be provided in the biosensor chip 1600 that controls the sensing operation of the MI sensors 1602 and the sensing circuits therein.

Those of skill in the art will further appreciate that the various illustrative logical blocks, modules, circuits, and algorithms described in connection with the aspects disclosed herein may be implemented as electronic hardware, instructions stored in memory or in another computer-readable medium and executed by a processor or other processing device, or combinations of both. The master devices and slave devices described herein may be employed in any circuit, hardware component, integrated circuit (IC), or IC chip, as examples. Memory disclosed herein may be any type and size of memory and may be configured to store any type of information desired. To clearly illustrate this interchangeability, various illustrative components, blocks, modules, circuits, and steps have been described above generally in terms of their functionality. How such functionality is implemented depends upon the particular application, design choices, and/or design constraints imposed on the overall system. Skilled artisans may implement the described functionality in varying ways for each particular application, but such implementation decisions should not be interpreted as causing a departure from the scope of the present disclosure.

The various illustrative logical blocks, modules, and circuits described in connection with the aspects disclosed herein may be implemented or performed with a processor, a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A processor may be a microprocessor, but in the alternating, the processor may be any processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The aspects disclosed herein may be embodied in hardware and in instructions that are stored in hardware, and may reside, for example, in Random Access Memory (RAM), flash memory, Read Only Memory (ROM), Electrically Programmable ROM (EPROM), Electrically Erasable Programmable ROM (EEPROM), registers, a hard disk, a removable disk, a CD-ROM, or any other form of computer readable medium known in the art. An exemplary storage medium is coupled to the processor such that the processor can read information from, and write information to, the storage medium. In the alternating, the storage medium may be integral to the processor. The processor and the storage medium may reside in an ASIC. The ASIC may reside in a remote station. In the alternating, the processor and the storage medium may reside as discrete components in a remote station, base station, or server.

It is also noted that the operational steps described in any of the exemplary aspects herein are described to provide examples and discussion. The operations described may be performed in numerous different sequences other than the illustrated sequences. Furthermore, operations described in a single operational step may actually be performed in a number of different steps. Additionally, one or more operational steps discussed in the exemplary aspects may be combined. It is to be understood that the operational steps illustrated in the flow chart diagrams may be subject to numerous different modifications as will be readily apparent to one of skill in the art. Those of skill in the art will also understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The previous description of the disclosure is provided to enable any person skilled in the art to make or use the disclosure. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein, but is to be accorded the widest scope consistent with the principles and novel features disclosed herein.

What is claimed is:

1. A magneto-impedance (MI) device, comprising:
a substrate; and
an MI structure, comprising:
a conducting layer disposed above the substrate, the conducting layer having a first contact area and a second contact area;
an insulating layer disposed above the conducting layer;
a ferromagnetic layer disposed above the insulating layer, the ferromagnetic layer comprising a bottom outer surface disposed adjacent to the insulating layer and a top outer surface; and
an exchange bias layer comprising an anti-ferromagnetic material disposed in contact with the top outer surface of the ferromagnetic layer.

2. The MI device of claim 1, further comprising:
a first electrode in electrical contact with the first contact area of the conducting layer; and
a second electrode in electrical contact with the second contact area of the conducting layer.

3. The MI device of claim 2, wherein the conducting layer is configured to generate magnetic flux in the ferromagnetic layer in response to an alternating current (AC) current flowing through the conducting layer from the first contact area to the second contact area.

4. The MI device of claim 3, wherein the insulating layer is configured to assist in confining the AC current within the conducting layer.

5. The MI device of claim 1, wherein the exchange bias layer is configured to pin interfacial magnetic moments of the ferromagnetic layer.

6. The MI device of claim 1, wherein the ferromagnetic layer has a magneto-impedance effect, wherein an impedance of the ferromagnetic layer is configured to change in a presence of an external magnetic field generated in the ferromagnetic layer.

7. The MI device of claim 1 encapsulated in an encapsulation material.

8. The MI device of claim 1, wherein the MI structure further comprises:
a second insulating layer disposed below the conducting layer;
a second ferromagnetic layer disposed below the insulating layer, the second ferromagnetic layer comprising a second top outer surface disposed adjacent to the second insulating layer and a second bottom outer surface; and
a second exchange bias layer comprising an anti-ferromagnetic material disposed in contact with the second top outer surface of the second ferromagnetic layer.

9. The MI device of claim 1, wherein the ferromagnetic layer comprises an amorphous ferromagnetic material.

10. The MI device of claim 9, wherein the amorphous ferromagnetic material is comprised from the group consisting of Cobalt (Co) Silicon (Si) Boron (B) (CoSiB), Co Iron (Fe) SiB (CoFeSiB), Nickel (Ni) Fe (NiFe), CoFeB, Co Fe Vanadium (V) B (CoFeVB), and CoFeSi Noobium (Nb) Copper (Cu) B (CoFeSiNbCuB).

11. The MI device of claim 1, wherein the insulating layer comprising an insulating material comprised from the group consisting of Silicon Oxide (SiO₂), Hafnium Oxide (HfOx), Magnesium Oxide (MgO), and Aluminum Oxide (AlOₓ).

12. The MI device of claim 1, wherein the conducting layer comprising a conducting material comprised from the group consisting of Copper (Cu), Silver (Ag), and Gold (Au).

13. The MI device of claim 1, wherein the exchange bias layer comprises the anti-ferromagnetic material comprised the group consisting of Iridium (Ir) Manganese (Mn) (IrMn), Platimum (Pt) Mn (PtMn), Nickel Oxide (NiO), and Cobalt Oxide (CoO).

14. The MI device of claim 7, wherein the encapsulation material is comprised from the group consisting of Silicon Oxide (SiO₂) and Silicon Nitride (SiN).

15. The MI device of claim 1, wherein:
the conducting layer has a thickness of approximately between 200-500 nanometers (nm);
the insulating layer has a thickness of approximately between 10-20 nm;
the ferromagnetic layer has a thickness of approximately between 100-200 nm; and
the exchange bias layer has a thickness of approximately between 5-25 nm.

16. The MI device of claim 1 having a total thickness of two (2) micrometers (μm) or less.

17. The MI device of claim 2,
wherein:
the MI structure is aligned along a longitudinal axis;
the MI structure comprises a first electrode and a second electrode; and
the first and second electrodes are aligned with one another along the longitudinal axis of the MI structure; and
further comprising a plurality of MI structures arranged with their respective longitudinal axes substantially in parallel with one another.

18. The MI device of claim 2, wherein the MI structure has a serpentine structure between the first and second contact areas of the conducting layer.

19. The MI device of claim 1, wherein:
the conducting layer comprises a sputtered conducting film material;
the insulating layer comprises a sputtered insulating film material;
the ferromagnetic layer comprises a sputtered ferromagnetic film material; and
the exchange bias layer comprises a sputtered anti-ferromagnetic film material.

20. The MI device of claim 1 integrated into an integrated circuit (IC) chip.

21. The MI device of claim 1 integrated into a device selected from the group consisting of: a wearable device, a point-of-care device, a bacterial infection diagnostic device, a cancer detection device, a heart disease diagnostic device, and a food safety monitoring device.

22. A magneto-impedance (MI) sensor, comprising:
an MI device encapsulated in an encapsulation material, the MI device comprising:
an MI structure, comprising:
a conducting layer disposed above a substrate, the conducting layer having a first contact area and a second contact area;
an insulating layer disposed above the conducting layer;
a ferromagnetic layer disposed above the insulating layer, the ferromagnetic layer comprising a bottom outer surface disposed adjacent to the insulating layer and a top outer surface; and
an exchange bias layer comprising an anti-ferromagnetic material disposed in contact with the top outer surface of the ferromagnetic layer;
a first electrode in electrical contact with the first contact area of the conducting layer; and
a second electrode in electrical contact with the second contact area of the conducting layer;
an external channel formed in a void in the encapsulation material, the external channel forming a biological area configured to capture magnetic nanoparticles;
an alternating current (AC) current source circuit electrically coupled to the first contact area and the second contact area of the conducting layer, the AC current source circuit configured to generate an AC current to flow through the conducting layer; and
a sensing circuit configured to:
receive a sense voltage of the conducting layer in response to the magnetic nanoparticles generating a magnetic stray field in the ferromagnetic layer and changing an impedance of the ferromagnetic layer; and
generate an output voltage based on the sense voltage representing the impedance of the ferromagnetic layer.

23. The MI sensor of claim 22, further comprising:
a second MI structure, comprising:
a second conducting layer having a first contact area and a second contact area;
a second insulating layer disposed above the second conducting layer;
a second ferromagnetic layer disposed above the second insulating layer, the second ferromagnetic layer comprising a second bottom outer surface disposed adjacent to the second insulating layer and a second top outer surface; and
a second exchange bias layer comprising a second anti-ferromagnetic material disposed in contact with the second top outer surface of the second ferromagnetic layer;
the sensing circuit further configured to:
receive a second sense voltage in the second ferromagnetic layer in response to the magnetic nanoparticles generating the magnetic stray field in the second ferromagnetic layer and changing an impedance of the second ferromagnetic layer; and
generate a second output voltage based on the second sense voltage representing the impedance of the second ferromagnetic layer; and
further comprising a sense amplifier configured to generate a differential output voltage indicative of a presence of the magnetic nanoparticles in the external channel based on a difference between the differential output voltage and the second output voltage.

24. The MI sensor of claim 23, wherein the external channel is disposed adjacent to the MI structure and the second MI structure, wherein the MI structure is disposed on a first side of the external channel and the second MI structure is disposed on a second side of the external channel substantially opposite the first side.

25. The MI sensor of claim 22 fabricated in a back-end-of-line (BEOL) of a complementary metal-oxide semiconductor (CMOS) integrated circuit (IC) chip.

26. The MI device of claim 22, wherein the external channel is configured to capture the magnetic nanoparticles bound to a bioreceptor bound to a target analyte of a biological sample.

27. A method of detecting a presence of magnetic nanoparticles in a magneto-impedance (MI) sensor, comprising:
receiving at least one magnetic nanoparticle configured to generate a magnetic stray field bound to a bioreceptor configured to capture a target analyte of interest in at least one external channel in an MI biosensor chip, each of the at least one external channel forming a biological active area, the MI biosensor chip comprising a plurality of MI devices each comprising:
a conducting layer disposed above a substrate, the conducting layer having a first contact area and a second contact area;
an insulating layer disposed above the conducting layer;
a ferromagnetic layer disposed above the insulating layer, the ferromagnetic layer comprising a bottom outer surface disposed adjacent to the insulating layer and a top outer surface; and
an exchange bias layer comprising an anti-ferromagnetic material disposed in contact with the top outer surface of the ferromagnetic layer;
generating an alternating current (AC) current to flow through the conducting layer to generate a magnetic flux in the ferromagnetic layer;
receiving a sense voltage in the ferromagnetic layer in response to the magnetic nanoparticles generating the magnetic stray field in the ferromagnetic layer and changing an impedance of the ferromagnetic layer; and
generating an output voltage based on the sense voltage representing the impedance of the ferromagnetic layer.

28. The method of claim 27, further comprising:
receiving a second sense voltage in a second ferromagnetic layer of a second MI device in response to the at least one magnetic nanoparticle generating the magnetic stray field in the second ferromagnetic layer and changing an impedance of the second ferromagnetic layer, the second MI device comprising:
a second conducting layer having a first contact area and a second contact area;
a second insulating layer disposed above the second conducting layer;
the second ferromagnetic layer disposed above the second insulating layer, the second ferromagnetic layer comprising a second bottom outer surface disposed adjacent to the second insulating layer and a second top outer surface; and
a second exchange bias layer comprising a second anti-ferromagnetic material disposed in contact with the second top outer surface of the second ferromagnetic layer;
receiving the second sense voltage in the second ferromagnetic layer in response to the at least one magnetic nanoparticle generating the magnetic stray field in the second ferromagnetic layer and changing the impedance of the second ferromagnetic layer;
generating a second output voltage based on the second sense voltage representing the impedance of the second ferromagnetic layer; and
generating a differential output voltage indicative of a presence of the at least one magnetic nanoparticle in the at least one external channel based on a difference between the differential output voltage and the second output voltage.

29. The method of claim 27, further comprising confining the AC current within the conducting layer.
